(19) United States
(12) Patent Application Publication
(10) Pub. No.: US 2004/0014109 A1
(43) Pub. Date: Jan. 22, 2004

(54) Methods and genes associated with screening assays for age at onset and common neurodegenerative diseases

(76) Inventors: Margaret A. Pericak-Vance, Chapel Hill, NC (US); Jeffery M. Vance, Chapel Hill, NC (US); Jonathan L. Haines, Brentwood, TN (US); John Gilbert, Oxford, NC (US); Yi-Ju Li, Durham, NC (US)

Correspondence Address: MYERS BIGEL SIBLEY & SAJOVEC, PO BOX 37428, RALEIGH, NC, 27627, US

(21) Appl. No.: 10/444,347

(22) Filed: May 23, 2003

Related U.S. Application Data
(60) Provisional application No. 60/382,880, filed on May 23, 2002.
(60) Provisional application No. 60/396,233, filed on Jul. 16, 2002.
(60) Provisional application No. 60/428,876, filed on Nov. 25, 2002.

(51) Int. Cl. C12Q 1/68

(52) U.S. Cl. 435/6

(57) ABSTRACT

The present invention discloses methods of screening a subject for Alzheimer's disease and/or Parkinson's disease comprising detecting the presence or absence of a marker or functional polymorphism associated with a gene linked to Alzheimer's disease and/or Parkinson's disease.

### CROSS REFERENCE TO RELATED APPLICATIONS

The present application claims priority to Provisional Application No. 60/382,880 filed May 23, 2002, Provisional Application No. 60/396,233 filed Jul. 16, 2002, and Provisional Application No. 60/428,876 filed Nov. 25, 2002, the disclosures of which are hereby incorporated by reference in their entireties.

### STATEMENT OF FEDERAL SUPPORT

This invention was made with support from the United States Federal government under grant numbers R01 NS31153, P50 NS39764, P01 NS26630, NS31153, AG05128, AG13308, AG11268, AG10123, AG19085, MH52453 and MH59528 from the National Institutes of Health. The United States Government has certain rights in this invention.

### FIELD OF THE INVENTION

This invention concerns methods of screening for Alzheimer's disease and/or Parkinson's disease by the screening of genetic risk factors.

### BACKGROUND OF THE INVENTION

Genetic studies of common complex neurodegenerative diseases, Such as Alzheimer's disease and Parkinson's disease, have focused on the identification of risk genes as targets for development of new treatments and improved diagnoses. This approach has identified the amyloid precursor protein (APP) (Goate et al., Nature 349:704-706 (1991)), presenilin 1 (PS1) (Sherrington et al., Nature 375:754-760 (1995)), presenilin 2 (PS2) (Levy-Lahad et al., Science 269:973-977 (1995); Rogaev et al., Nature 376:775-778 (1995)), and apolipoprotein E (APOE) (Corder et al., Science 261:921-923 (1993)) genes as contributing to risk in Alzheimer's disease. APP, PS1, and PS2 cause rare early-onset autosomal dominant Alzheimer's disease (5% of Alzheimer's disease cases), whereas APOE is associated with both risk and age at onset (AAO) in late-onset familial Alzheimer's disease, as well as in late- and early-onset sporadic Alzheimer's disease. Similarly, three genes have been identified to associate with risk in Parkinson's disease: α-synuclein (Polymeropoulos et al., Science 274:1197-1199 (1996)) for rare autosomal dominant early-onset Parkinson's disease, Parkin (Abbas et al., Hum Mol Genet 8:567-574 (1999)) for rare autosomal recessive juvenile parkinsonism and autosomal recessive early-onset Parkinson's disease, and tau (Martin et al., JAMA 286:2245-2250 (2001)) for classic Parkinson's disease. Genomic screens in both Parkinson's disease (Destefano et al., Neurology 57:1124-1126 (2001); Scott et al., JAMA 286:2239-2244 (2001)) and Alzheimer's disease (Kehoe et al., Hum Mol Genet 8:237-245 (1999); Pericak-Vance et al., Exp Gerontol 35:1343-1352 (2000) have recently localized additional but, as yet, unknown risk genes.

Both Alzheimier's disease and Parkinson's disease are neurodegenerative, late-AAO disorders. Clinically, a significant number of patients with Alzheimer's disease develop signs of parkinsonism, including bradykinesia, rigidity, and gait abnormalities. Conversely, dementia is a major factor in Parkinson's disease, and the two disorders both exhibit degeneration of cholinergic neurons in the nucleus basalis of Meynert. Another pathological similarity is the presence of similar staining α-synuclein Lewy bodies (LB) in both disorders, supporting the premise that the two diseases may share a common pathway leading to LB formation (See, Lippa et al., Arch Neurol 58:1817-1820 (2001)). APOE, a well-proven risk factor for Alzheimier's disease, has been suggested to be involved in the risk for Parkinson's disease as well, although others have failed to confirm these findings. Khan et al., Ann Neurol 49:665-668 (2001). The tau gene, a major element in the neurofibulary tangles of Alzheimer's disease, has recently been shown to be associated with Parkinson's disease as well (See, Martin et al.). These similarities are likely not derived from the biologic events that initiate the start of each disease but rather from overlapping neuropathic pathways involved in the progression of each disorder.

Several recent reports have focused attention on chromosome 10q for Alzheimer's disease, (Bertram et al., Science 290:2302-2305 (2000); Ertekin-Taner et al., Science 290:2303-2304 (2000); Myers et al., Science 290:2304-2305 (2000); Pericak-Vance) but have been inconsistent in localization (FIG. 2). Only the region identified by Bertram et al. (2000) by use of the NIMH family-sample data set maps near the present region. FIG. 2 depicts the map positions of published linkage regions for risk genes on chromosome 10q and the present linkage Findings for AAO genes. The linkage effect on chromosome 10q spans the different data sets used in the analyses. Three independent risk-gene studies (Ertekin-Taner et al.; Myers et al.; and Pericak-Vance et al.) identified an overlapping region (between D10S1225 and D10S1211) that is ˜47 cM proximal to the present linkage region for AAO. For Parkinson's disease, no risk genes have yet been reported on chromosome 10. See, Scott et al. 2001.

GSTO1 (or called GSTO1-1) is a member of the glutathione S-transferase (GST) family of genes that utilize glutathione in reactions and contribute to the biotransformation and disposition of many compounds, including drugs, carcinogens, and the products of oxidative stress. GSTO1 is known to be expressed in a wide range of tissues, specifically neural glial cells Board et al., J Biol. Chem. 275, 24798 (2000). GSTO1 is unusual in that it has a different substrate profile than other members of this family, notably a glutathione dependent thiotransferase activity similar to glutaredoxins. In addition, Dulhunty et al. have reported that in cells containing ryanodine receptors it may have a role in protecting from calcium-induced apotosis. J. Biol. Chem. 276, 3319 (2001). Zakharyan et al. have implicated it in the biotransformation of inorganic arsenic as well. Chem. Res. Toxicol. 14, 1051 (2001).

Identification of further genes would open new avenues of research with the potential to delay onset beyond the natural life span. Present knowledge about genes contributing to AAO in neurodegenerative diseases clearly lags behind the understanding of genes contributing to risk. Recently, there has been growing interest in using AAO information as a quantitative trait, to identify genes that influence onset of disease (Daw et al., Am J Hum Genet 64:839-851 (1999), Daw et al., Am J Hum Genet 66:196-204 (2000); Duggirala et al. Am J Hum Genet 64:1127-1140 (1999)). Rapid development of methods of mapping quantitative trait loci (QTLs) for general pedigrees (Goldgar, Am J Hum Genet 47:957-967 (1990); Amos, Am J Hum Genet 54:535-543 (1994); Blangero et al. Genet Epidemiol 14:959-964 (1997)) has now made the search for novel genes affecting AAO feasible. Nevertheless, the genetic basis for Alzheimer's disease and Parkinson's disease is not well understood, and there is a continued need to develop new genetic linkages and markers as well as identifying new functional polymorphisms that are associated with Alzheimer's disease and Parkinson's disease. Because Alzheimer's disease and Parkinson's disease share some common clinical and pathological findings, it may be advantageous to locate a gene or genes common to both disorders.

### SUMMARY OF THE INVENTION

The present invention discloses methods of screening a subject for Alzheimer's disease and/or Parkinson's disease. The method comprises the steps of: detecting the presence or absence of a marker for Alzheimer's disease and/or Parkinson's disease, or a functional polymorphism associated with a gene linked to Alzheimer's disease and/or Parkinson's disease, with the presence of such a marker or functional polymorphism indicating that subject is afflicted with or at risk of developing Alzheimer's disease and/or Parkinson's disease. The detecting step may include detecting whether the subject is heterozygous or homozygous for the marker and/or functional polymorphism, with subjects who are at least heterozygous for the functional polymorphism being at increased risk for Alzheimer's disease and/or Parkinson's disease. The step of detecting the presence or absence of the marker or functional polymorphism may include the step of detecting the presence or absence of the marker or functional polymorphism in both chromosomes of the subject (i.e., detecting the presence or absence of one or two alleles containing the marker or functional polymorphism). More than one copy of a marker or functional polymorphism (i.e., subjects homozygous for the functional polymorphism) may indicate greater risk of Alzheimer's disease and/or Parkinson's disease as compared to heterozygous subjects.

A further aspect of the present invention is the use of a means of detecting a marker, functional polymorphism or mutation as described herein in screening a subject for Alzheimer's disease and/or Parkinson's disease as described herein.

The foregoing and other objects and aspects of the present invention are explained in detail in the drawings herein and the specification set forth below.

### BRIEF DESCRIPTION OF THE DRAWINGS

FIG. 1 is a graph depicting the results of chromosome 10 multipoint linkage analyses.

FIG. 2 is a chart illustrating the map positions of reported linkage results for the risk of AAO genes on chromosome 10q.

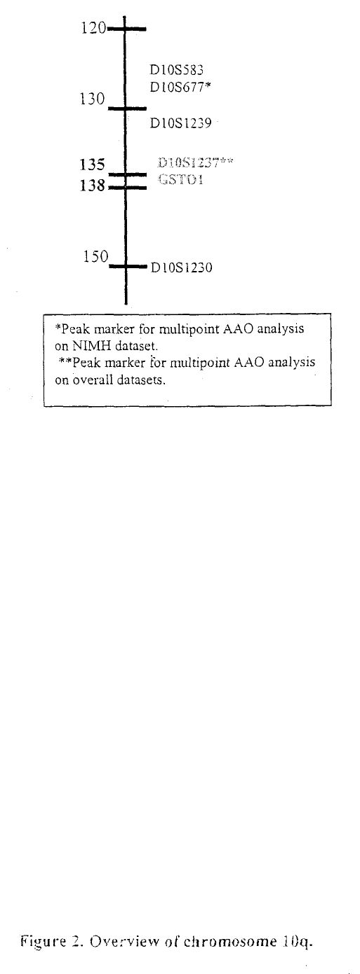

FIG. 3 illustrates the locations of candidate genes used in the present study in relationship to the linkage region.

FIG. 4 is a graph depicting P-values derived from the orthogonal model for all SNPs genotyped in the Alzheimer's disease dataset.

### DETAILED DESCRIPTION OF THE EMBODIMENTS

As noted above, the present invention provides a method of screening (e.g., diagnosing, detecting, determining or prognosing) for Alzheimer's disease and/or Parkinson's disease in a subject. Subjects with which the present invention is concerned are primarily human subjects, including male and female subjects of any age or race.

The term “Alzheimer's disease” (AD) as used herein is intended to encompass all types of Alzheimer's disease, including sporadic and familial Alzheimer's disease, as well as late onset and early onset Alzheimer's disease.

The term “late-onset Alzheimer's disease” refers to Alzheimer's disease which has a time of onset after the subject reaches 60 years of age.

The term “age at onset” (AAO) refers to the age at which a subject is affected with a particular disease.

The term “Parkinson's disease” (PD) as used herein is intended to encompass all types of Parkinson's disease.

“Screening” as used herein refers to a procedure used to evaluate a subject for risk of Alzheimer's disease and/or Parkinson's disease. It is not required that the screening procedure be free of false positives or false negatives, as long as the screening procedure is useful and beneficial in determining which of those individuals within a group or population of individuals are at increased risk of Alzheimer's disease and/or Parkinson's disease. A screening procedure may be carried out for both prognostic and diagnostic purposes (i.e., prognostic methods and diagnostic methods).

“Prognostic method” refers to methods used to help predict, at least in part, the course of a disease. For example, a screening procedure may be carried out on a subject that has not previously been diagnosed with Alzheimer's disease and/or Parkinson's disease, or does not show substantial disease symptoms, when it is desired to obtain an indication of the future likelihood that the subject will be afflicted with Alzheimer's disease and/or Parkinson's disease. In addition, a prognostic method may be carried out on a subject previously diagnosed with Alzheimer's disease and/or Parkinson's disease when it is desired to gain greater insight into how the disease will progress for that particular subject (e.g., the likelihood that a particular patient will respond favorably to a particular drug treatment, or when it is desired to classify or separate Alzheimer's disease and/or Parkinson's disease patients into distinct and different subpopulations for the purpose of conducting a clinical trial thereon). A prognostic method may also be used to determine whether a person will respond to a particular drug.

“Diagnostic method” as used herein refers to screening procedures carried out on a subject that has previously been determined to be at risk for a particular neurodegenerative disorder due to the presentation of symptoms or the results of another (typically different) screening test.

“Functional polymorphism” as used herein refers to a change in the base pair sequence of a gene that produces a qualitative or quantitative change in the activity of the protein encoded by that gene (e.g., a change in specificity of activity; a change in level of activity). The presence of a functional polymorphism indicates that the subject is at greater risk of developing a particular disease as compared to the general population. For example, the patient carrying the functional polymorphism may be particularly susceptible to chronic exposure to environmental toxins that contribute to Alzheimer's disease and/or Parkinson's disease. The term “functional polymorphism” includes mutations, deletions and insertions.

A “present” functional polymorphism as used herein (e.g., one that is indicative of or a risk factor for Alzheimer's disease and/or Parkinson's disease) refers to the nucleic acid sequence corresponding to the functional polymorphism that is found less frequently in the general population relative to Alzheimer's disease and/or Parkinson's disease as compared to the alternate nucleic acid sequence or sequences found when such functional polymorphism is said to be “absent”.

“Mutation” as used herein sometimes refers to a functional polymorphism that occurs in less than one percent of the population, and is strongly correlated to the presence of a gene (i.e., the presence of such mutation indicating a high risk of the subject being afflicted with a disease). However, “mutation” is also used herein to refer to a specific site and type of functional polymorphism, without reference to the degree of risk that particular mutation poses to an individual for a particular disease.

“Linked” as used herein refers to a region of a chromosome that is shared more frequently in family members affected by a particular disease, than would be expected by chance, thereby indicating that the gene or genes within the linked chromosome region contain or are associated with a marker or functional polymorphism that is correlated to the presence of, or risk of, disease. Once linkage is established association studies (linkage disequilibrium) can be used to narrow the region of interest or to identify the risk conferring gene for Alzheimer's disease and/or Parkinson's disease.

“Associated with” when used to refer to a marker or functional polymorphism and a particular gene means that the functional polymorphism is either within the indicated gene, or in a different physically adjacent gene on that chromosome. In general, such a physically adjacent gene is on the same chromosome and within 2, 3, 5, 10 or 15 centimorgans of the named gene (i.e., within about 1 or 2 million base pairs of the named gene). The adjacent gene may span over 5, 10 or even 15 megabases.

A unit of measure of recombination frequency. One centimorgan is equal to a 1% chance that a marker at one genetic locus will be separated from a marker at a second locus due to crossing over in a single generation. In humans, 1 centimorgan is equivalent, on average, to one million base pairs.

Markers (e.g., genetic markers such as restriction fragment length polymorphisms and simple sequence length polymorphisms) may be detected directly or indirectly. A marker may, for example, be detected indirectly by detecting or screening for another marker that is tightly linked (e.g., is located within 2 or 3 centimorgans) of that marker. Additionally, the adjacent gene may be found within an approximately 15 cM linkage region surrounding the chromosome thus spanning over 5, 10 or even 15 megabases.

The presence of a marker or functional polymorphism associated with a gene linked to Alzheimer's disease and/or Parkinson's disease indicates that the subject is afflicted with Alzheimer's disease and/or Parkinson's disease or is at risk of developing Alzheimer's disease and/or Parkinson's disease. A subject who is “at increased risk of developing Alzheimer's disease and/or Parkinson's disease” is one who is predisposed to the disease, has genetic susceptibility for the disease or is more likely to develop the disease than subjects in which the detected functional polymorphism is absent. While the methods described herein may be employed to screen for any type of idiopathic Alzheimer's disease and/or Parkinson's disease, a primary application is in screening for late-onset Alzheimer's disease and/or early-onset Parkinson's disease.

The marker or functional polymorphism may also indicate “age of onset” of Alzheimer's disease and/or Parkinson's disease, particularly subjects at risk for Alzheimer's disease and/or Parkinson's disease, with the presence of the marker indicating an earlier age of onset for Alzheimer's disease and/or Parkinson's disease.

Suitable subjects include those who have not previously been diagnosed as afflicted with Alzheimer's disease and/or Parkinson's disease, those who have previously been determined to be at risk of developing Alzheimer's disease and/or Parkinson's disease, and those who have been initially diagnosed as being afflicted with Alzheimer's disease and/or Parkinson's disease where confirming information is desired. Thus, it is contemplated that the methods described herein be used in conjunction with other clinical diagnostic information known or described in the art which are used in evaluation of subjects with Alzheimer's disease or suspected to be at risk for developing such disease.

The detecting step may be carried out in accordance with known techniques (See, e.g., U.S. Pat. Nos. 6,027,896 and 5,508,167 to Roses et al.), such as by collecting a biological sample containing DNA from the subject, and then determining the presence or absence of DNA encoding or indicative of the functional polymorphism in the biological sample (e.g., the Parkin gene exon 3 deletion mutation described herein). Any biological sample which contains the DNA of that subject may be employed, including tissue samples and blood samples, with blood cells being a particularly convenient source.

Determining the presence or absence of DNA encoding a particular functional polymorphism may be carried out with an oligonucleotide probe labeled with a suitable detectable group, and/or by means of an amplification reaction such as a polymerase chain reaction or ligase chain reaction (the product of which amplification reaction may then be detected with a labeled oligonucleotide probe or a number of other techniques). Further, the detecting step may include the step of detecting whether the subject is heterozygous or homozygous for the particular functional polymorphism. Numerous different oligonucleotide probe assay formats are known which may be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (applicants specifically intend that the disclosures of all U.S. Patent references cited herein be incorporated herein by reference).

Amplification of a selected, or target, nucleic acid sequence may be carried out by any suitable means. See generally, Kwoh et al., Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad Sci. USA 86, 1173-1177 (1989)), self-sustained sequence replication (or “3SR”) (see J. Guatelli et al., Proc. Natl. Acad. Sci. USA 87, 1874-1878 (1990)), the Qβ replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). Polymerase chain reaction is currently preferred.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybricizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. When PCR conditions allow for amplification of all allelic types, the types can be distinguished by hybridization with an allelic specific probe, by restriction endonuclease digestion, by electrophoresis on denaturing gradient gels, or other techniques.

DNA amplification techniques such as the foregoing can involve the use of a probe, a pair of probes, or two pairs of probes which specifically bind to DNA containing the functional polymorphism, but do not bind to DNA that does not contain the functional polymorphism. Alternatively, the probe or pair of probes could bind to DNA that both does and does not contain the functional polymorphism, but produce or amplify a product (e.g., an elongation product) in which a detectable difference may be ascertained (e.g., a shorter product, where the functional polymorphism is a deletion mutation). Such probes can be generated in accordance with standard techniques from the known sequences of DNA in or associated with a gene linked to Alzheimer's disease and/or Parkinson's disease or from sequences which can be generated from such genes in accordance with standard techniques.

It will be appreciated that the detecting steps described herein may be carried out directly or indirectly. Other means of indirectly determining allelic type include measuring polymorphic markers that are linked to the particular functional polymorphism, as has been demonstrated for the VNTR (variable number tandem repeats) and the ApoB alleles (Decorter et al., DNA & Cell Biology 9(6), 461-69 (1990), and collecting and determining differences in the protein encoded by a gene containing a functional variant, as described for ApoE4 in U.S. Pat. Nos. 5,508,167 and 6,027,896 to Roses et al.

Kits for determining if a subject is or was (in the case of deceased subjects) afflicted with or is or was at increased risk of developing Alzheimer's disease will include at least one reagent specific for detecting for the presence or absence of at least one functional polymorphism as described herein and instructions for observing that the subject is or was afflicted with or is or was at increased risk of developing Alzheimer's disease if at least one of the functional polymorphisms is detected. The kit may optionally include one or more nucleic acid probes for the amplification and/or detection of the functional polymorphism by any of the techniques described above, with PCR being currently preferred.

Alzheimer's disease and Parkinson's disease, although distinct clinical entities, share some common clinical and pathological features. Both have substantial variability in AAO. Although Lewy bodies are a cardinal feature of Parkinson's disease, they can be found in individuals with autopsy-confirmed Alzheimer's disease (Hulette et al. 2000; Scott et al., Am J Hun Genet 66:922-932 (2000)). Dementia is the primary feature of Alzheimer's disease, but a substantial number of patients with Parkinson's disease also develop dementia as their Parkinson's disease progresses. See Scott et al. 2001. Thus, it may be possible that Alzheimer's disease and Parkinson's disease share common etiologic pathways.

The present invention may use the variance-component procedure in SOLAR to perform genomewide scans on the quantitative trait AAO for Alzheimer's disease and Parkinson's disease to map quantitative trait loci influencing AAO. The present method may be less penetrance-model dependent than the classical segregation/linkage-mapping technique, thus, it may take into account, covariate or random effects. The common regions showing evidence of linkage from independent analyses of Alzheimer's disease and Parkinson's disease data sets may be further analyzed by use of the combined Alzheimer's disease and Parkinson's disease data set (ADPD).

Genomic screens have concentrated historically on identifying genes controlling the risk of developing a disease. However, risk is not the only important aspect of a disease. Onset of disease is also crucial, as understanding the regulation of onset could make it possible to delay onset beyond an individual's normal life span. The results as discussed below demonstrate that AAO is highly heritable and that the search for AAO genes is possible. It should be noted that AAO data are very difficult to acquire reliably, and false-negative results may be produced. The large sample sizes assembled in the present study for both Alzheimer's disease and Parkinson's disease should help to decrease the false-negative outcome.

The present genomic screen for AAO in Alzheimer's disease has identified several linkage regions for AAO, in which chromosomes 4q, 8q, and 10q show the most promising results, with LOD scores >2. The APOE gene still yielded the strongest linkage effect among the newly identified regions in Alzheimer's disease, and the role of APOE in controlling onset of Alzheimer's disease was further confirmed. For Parkinson's disease, a single peak was identified with very strong linkage evidence (LOD=3.41) near D1S2134 (78 cM). Previously, Valente et al., Am J Hum Genet 68:895-900 (2001) and van Duijn et al., Am J Hum Genet 69:629-634 (2001) had localized genes for rare autosomal recessive early-onset Parkinson's disease to two independent regions on chromosome 1p (PARK6 and PARK7, respectively). The minimal candidate region (MCR) for PARK6 defined by the observed recombination in the family is between D1S483 (45.3 cM) and D1S247 (57.8 cM), whereas the MCR for PARK7 is between D1S468 (4.2 cM) and DIS214 (14 cM). In addition, the weak support for linkage on chromosome 1q (LOD=1.20 at 214 cM) to Parkinson's disease risk was previously reported and is unlinked to the present region. See, DeStefano et al. supra.

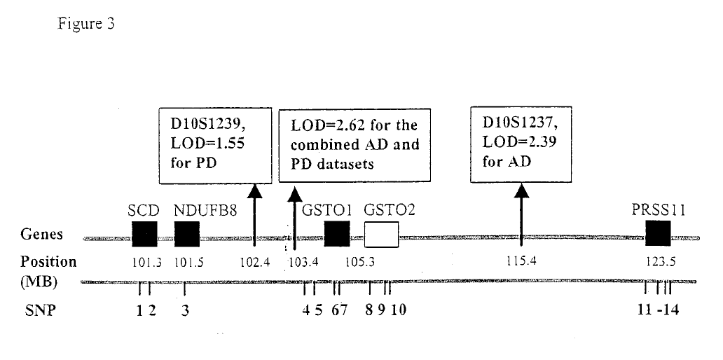

The present invention also includes the discovery of a ˜15 cM linkage region surrounding D10S1239 and D10S1237 on chromosome 10q (FIG. 3). FIG. 3 illustrates the locations of candidate genes used in the present study in relationship to the linkage region. The solid black boxes indicate the genes identified by microarray study. The tick marks represent the SNPs genotyped in this study. Their actual position is listed in Table S1 listed below. The physical position is in megabases (MB).This linkage region was found in both Alzheimer's disease and Parkinson's disease families, suggesting the existence of a common gene affecting age-at-onset in both disorders. This linkage peak is large, spanning over 15 megabases. Thus the concept of “genomic convergence” was adopted to prioritize the evaluation of candidate genes by integrating genome-wide gene expression data with the existing genome-wide linkage screen data. Hauser et al., Hum. Mol. Genet. 12, 671 (2003).

The gene expression study was conducted by isolating human brain RNA from the hippocampus of six Alzheimer's disease patients and two matched controls. Alzheimer's disease patients were in Braak and Braak stage IV or V and normal controls were in Braak and Braak stage I. Alzheimer's disease patients of APOE4/4, APOE4/3, and APOE3/3 genotypes were chosen along with controls of the APOE 3/3 genotype. The post-mortem delay ranges from 1 to 17 hours for Alzheimer's disease patients and 4 to 10 hours for controls. The brain tissues were collected in the Alzheimer's Disease Research Center, Duke University Medical Center. See, Braak, Brain Pathol., 1, 213 (1991); and Braak, Int. Psychogeriatr., 9 Suppl 1, 257 (1997). The cRNA was hybridized to microarrays (Affymetrix GeneClips U133A array) containing oligonucleotide probe-sets with 22,000 predicted or known human genies. Fifty-two genes demonstrated significant differences in gene expression levels between Alzheimer's disease and controls.

Four of these genes map to the chromosome 10q linkage region: Stearoyl-CoA desaturase (SCD; MIM 604031); NADH-ubiquinone oxidoreductase 1 beta complex, 8 (NDUFB8; MIM 602140); glutatlionie S-transferase, omega-1 (GSTO1; MIM 605482); and protease, serine 11 (PRSS11; MIM 602194). The transcripts of these four genes were significantly down regulated in Alzheimer's disease in comparison to controls (Table 1).

TABLE 1
List of genes in chromosome 10 q showing significant differential
expression between Alzheimer's disease and controls.

| Affymetrix | | | |
|---|---|---|---|
| Probe ID | AD/Control* | p-value | description |
| 200832_s_at | 0.518 | 0.039 | Stearoyl-CoA desaturase (SCD) |
| 201185_at | 0.540 | 0.0009 | Protease, serine, 11 (PRSS11) |
| | | | NADH-ubiquinone oxido- |
| | | | reductase 1 beta |
| 201227_s_at | 0.627 | 0.027 | complex, 8 (NDUFB8) |
| 201470 at | 0.626 | 0.029 | glutathione-S-transferase, |
| | | | omega-I (GSTO1) |

*Average raw intensity in each group was used.

FIG. 3 depicts the genomic locations in relationship to D10S1239 and D10S1237, linkage peaks for Parkinson's disease and Alzheimer's disease, respectively. The primary candidate gene is GSTO1, located between D10S1239 and D10S1237 and under the linkage peak (LOD=2.62) of the combined Alzheimer's disease and Parkinson's disease dataset. Upon evaluation of this genomic region, a second defined actively transcribed member of the glutathione transferase omega class, GSTO2, which lies 7.5 kb downstream of GSTO1 and was not on the Affymetrix GeneChip U133A array. GSTO2 has 64% amino acid identity with GSTO1. Allelic association was tested between SNPs in these five candidate genes and age-at-onset in Alzheimer's disease. The Alzheimer's disease dataset includes 606 multiplex and 105 discordant sibpair families. Clinical diagnosis was based on consensus criteria. See, McKhann et al., Neurol. 34, 939 (1984). At least one individual in each of the 711 families has Alzheimer's disease diagnosis of either “definite” (autopsy confirmed) or “probable” (clinically affected without autopsy). The genes showing significant results in Alzheimer's disease were followed up using the Parkinson's disease dataset. The Parkinson's disease dataset consists of 282 multiplex and 16 discordant sibling pair families with a total of 635 Parkinson's disease patients. Diagnosis for Parkinson's disease patients followed consensus criteria. See, Scott et al., JAMA 286, 2239 (2001).

Fourteen single nucleotide polymorphisms was genotyped spanning these five candidate genes in the 1773 Alzheimer's disease patients and 1041 relatives. Strong linkage disquilibrium (LD) was seen between pairs of SNPs in the region between SNP4 and SNP 10 (within GSTO1 and GSTO2) (Table S1).

TABLE S1
Pairwise Pearson correlation (r²) for all SNPs genotyped in the
Alzheimer's disease dataset. The lower triangle is for unaffected group and upper triangle is
for affected group.

| | Mark- | SNP | SNP | SNP | SNP | SNP | SNP | SNP | SNP | SNP | SNP | SNP | SNP | SNP | SNP |
|---|---|---|---|---|---|---|---|---|---|---|---|---|---|---|---|
| Gene | er | 1 | 2 | 3 | 4 | 5 | 6 | 7 | 8 | 9 | 10 | 11 | 12 | 13 | 14 |
| SCD | SNP1 | | 0.46 | 0.013 | 0 | 0 | 0 | 0.004 | 0 | 0.002 | 0.005 | 0 | 0.002 | 0.004 | 0.006 |
| | SNP2 | 0.432 | | 0.01 | 0 | 0 | 0.001 | 0.004 | 0.001 | 0.001 | 0.003 | 0 | 0 | 0.003 | 0.001 |
| NDUFB8 | SNP3 | 0.018 | 0.01 | | 0 | 0 | 0 | 0 | 0 | 0.002 | 0 | 0.005 | 0.005 | 0.003 | 0.005 |
| GSTO1 | SNP4 | 0.002 | 0.004 | 0.001 | | 0.592 | 0.56 | 0.387 | 0.498 | 0.202 | 0.336 | 0.001 | 0.013 | 0 | 0.001 |
| | SNP5 | 0 | 0.01 | 0 | 0.686 | | 0.795 | 0.661 | 0.73 | 0.368 | 0.596 | | | | |
| | | | | | | | | | | | | 0.001 | 0.013 | 0.001 | 0.001 |
| | SNP6 | 0.004 | 0.009 | 0.003 | 0.608 | 0.749 | | 0.723 | 0.893 | 0.386 | 0.628 | 0 | 0.008 | 0 | 0 |
| | SNP7 | 0 | 0 | 0 | 0.482 | 0.684 | 0.76 | | 0.65 | 0.562 | 0.899 | 0.002 | 0.005 | 0.002 | 0 |
| GSTO2 | SNP8 | 0.004 | 0.012 | 0.001 | 0.519 | 0.662 | 0.888 | 0.671 | | 0.469 | 0.723 | 0 | 0.009 | 0 | 0 |
| | SNP9 | 0 | 0.003 | 0.004 | 0.222 | 0.353 | 0.389 | 0.522 | 0.488 | | 0.65 | 0.002 | 0.002 | 0.002 | 0 |
| | SNP10 | 0 | 0.001 | 0.001 | 0.416 | 0.608 | 0.657 | 0.872 | 0.769 | 0.631 | | 0.001 | 0.005 | 0.003 | 0 |
| PRSS11 | SNP11 | 0.001 | 0.012 | 0.005 | 0 | 0 | 0.001 | 0.008 | 0.001 | 0.005 | 0.004 | | 0.172 | 0.14 | 0.209 |
| | SNP12 | 0.011 | 0.013 | 0.004 | 0 | 0.001 | 0 | 0.001 | 0 | 0.005 | 0.001 | 0.129 | | 0.057 | 0.085 |
| | SNP13 | 0 | 0.015 | 0 | 0.001 | 0 | 0 | 0.004 | 0 | 0.001 | 0.005 | 0.124 | 0.066 | | 0.498 |
| | SNP14 | 0.009 | 0.015 | 0 | 0 | 0 | 0.005 | 0.001 | 0.004 | 0 | 0 | 0.22 | 0.1 | 0.46 | |

*Bold numbers represent strong LD in both the affected and unaffected groups.

Other SNPs were not in LD with any SNPs located in GSTO1 and GSTO2 indicating that GSTO1 and GSTO2 form an independent LD group.

Age-at-onset was analyzed as a quantitative trait. The mean age-at-onset±SD was 71.5±8 years (range: 40-97 years) for the Alzheimer's disease dataset and 59±13 years (range: 12-90 years) for the Parkinson's disease dataset. The orthogonal model was used which applies to general pedigrees, to test allelic association between each SNP and age-at-onset. See, Abecasis et al., Eut. J. Hum. Genet., 8, 545 (2000). To examine the direction of the effect on age-at-onset, the Monks-Kaplan method was applied which uses parental and sibpair data only. See, Monks et al., Am. J. Hum. Genet. 66, 576 (2000). A conservative Bonferroni correction was utilized for multiple testing with a nominal significant p-value of 0.05 results in p=0.004 as a threshold to declare statistical significance for the Alzheimer's disease dataset.

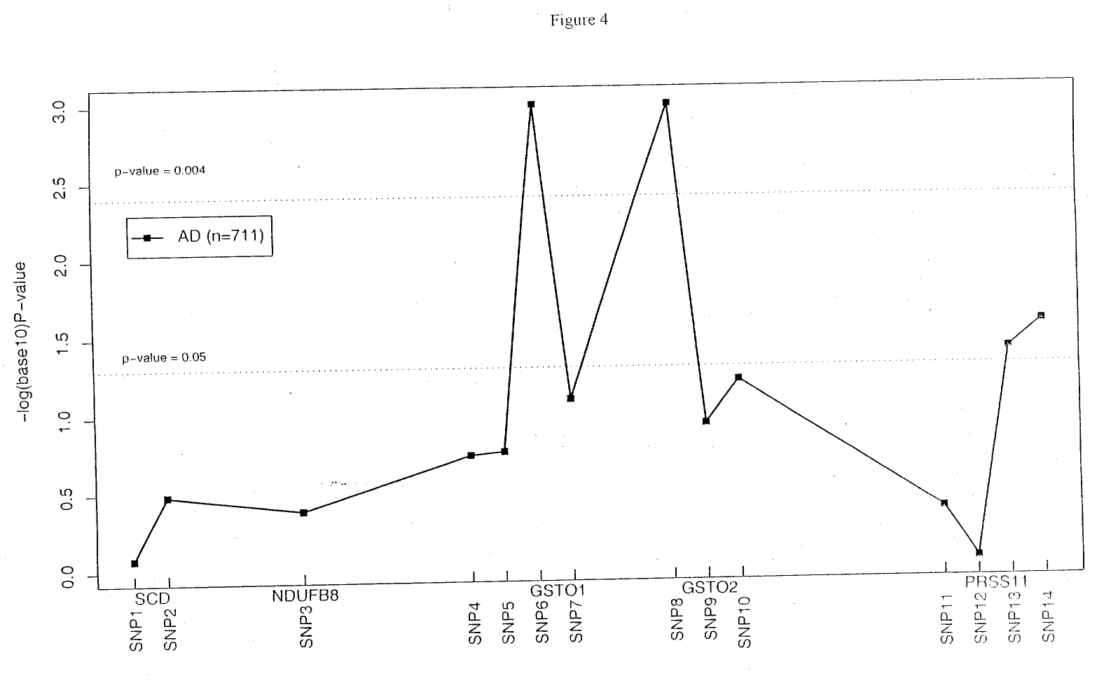

The p-values obtained from the orthogonal model (OM) for all SNPs are depicted in FIG. 4. Both GSTO1 SNP6 and GSTO2 SNPS are significantly associated with age-at-onset in Alzheimer's disease (P=0.001). SNP13 and SNP14 in PRSS11 showed interesting p-values (P<0.05), but these OM findings were not confirmed by the Monks-Kaplan method (MNK). Independent examination of SNP6 and SNP8 in the Parkinson's disease dataset using both OM and MK confirmed the above findings (P=0.026 (OM), 0.028 (MK) for SNP6; P=0.042 (OM and MK) for SNP8). The combined Alzheimer's disease and Parkinson's disease datasets generated similar results for both SNPs (P<0.004), indicating that there may be at least one common mechanism underlying age-at-onset in neurodegenerative disease. SNP6 (Ala140Asp) in GSTO1 causes a non-conservative amino acid change from a hydrophobic to a hydrophilic residue. The MK method detected that the less common asparagine (Asp) allele for GSTO1 SNP6 and the −183C allele for GSTO2 SNP8 are associated with later, and thus delayed, age at onset for both Alzheimer's disease and Parkinson's disease.

Since apolipoprotein E (APOE), particularly the APOE-4 allele, is associated with age-at-onset in Alzheimer's disease, and more recently in Parkinson's disease, the effect of APOE on these SNPs was additionally further examined. The inclusion of APOE as a covariate had little effect on the association results (p-values remained similar for each SNP), suggesting that the association between the SNPs and age-at-onset is independent from any APOE effect.

The similar association pattern seen with age-at-onset in SNP6 (GSTO1) and SNP8 (GSTO2) would appear to be due to the strong LD between these two SNPs. Since the SNPs in GSTO1 and GSTO2 are not in LD with those in SCD, NDUFB8, or PRSS11, and the SNPs located upstream of SNP6 or downstream of SNP8 are not associated with age-at-onset in Alzheimer's disease, the region in GSTO1 and GSTO2 flanked by SNP6 and SNP8 is the primary candidate region for a susceptibility allele for age-at-onset in Alzheimer's disease.

To determine whether this allelic association might be detecting a risk effect rather than a clear modifier of age-at-onset, GSTO1 was tested for risk to Alzheimer's disease and Parkinson's disease by using the pedigree disequilibrium test (PDT), in which the disease phenotype is the trait of interest. Martin et al., Am. J. Hum. Genet. 67, 146 (2000). There was no evidence for allelic association of any of the SNPs with risk of developing either disease. Therefore the effect of GSTO1 appears to be specific to age-at-onset.

Recently, the Asp allele of SNP6 was shown to result in a 25% decrease on thioltransferase activity of GSTO1, suggesting its location can affect the protein's function. SNP8 is 183 bp upstream of exon 1 of GSTO2, and could conceivably have an effect on this gene's promoter. Tanaka-Kagawa et al., Biochem. Biophys. Res. Commun. 301, 516 (2003). However, the lack of association with SNP9 and SNP10 located in intron 3 and exon 4 of GSTO2, the strong LD between SNP6 and SNP8, and Tanaka-Kagawa et al's recent report of functional association with GSTO1 Ala 140Asp polymorphism support GSTO1, rather than GSTO2, as the primary candidate gene affecting age-at-onset of Alzheimer's disease and Parkinson's disease. The evidence for GSTO1 affecting age-at-onset is strengthened by the present finding that APOE did not affect the association identified.

The present invention is explained in greater detail in the Examples that follow. These examples are intended as illustrative of the invention and are not to be taken as limiting thereof.

### EXAMPLES

Family Ascertainment

The data from numerous families with Alzheimer's disease and Parkinson's disease were studied to identify genetic risk factors. Specifically, 449 families affected with Alzheimer's disease and 174 families affected with Parkinson's disease were used as a test group. In all data sets, affected individuals were classified in accordance with the National Institute of Neurological and Communicative Disorders and Stroke and the Alzheimer's Disease and Related Disorders Associations (NINCDS-ADRDA) clinical diagnostic criteria. See, McKhann et al., Neurology 34: 939-944 (1984). The reported AAO of patients with Alzheimer's disease was defined as the age at which the caregiver, family, and/or individual first noted cognitive problems (most often short-term memory loss and, more rarely, other problems, such as dysphasia or disorientation to time or place, followed closely by memory change) sufficient to interfere with independent daily activities. The data from families with Parkinson's disease were ascertained by 13 centers in the United States and Australia. Diagnostic and exclusion criteria, based on previously published diagnostic criteria for Parkinson's disease, were adopted by all participating clinicians before selection of families began. See, e.g., Ward et al., Adv Neurol 53: 245-249 (1990); Hughes et al. Neurology 42:1142-1146 (1992a), J Neurol Neurosurg Psychiatry 55:181-184 (1992b). Affected individuals were defined by having at least two cardinal signs of Parkinson's disease (e.g., rest tremor, bradykinesia, and rigidity) and no atypical clinical features or other causes of parkinsonism. The reported AAO was defined as the age at which an affected individual first noticed one of the cardinal signs of Parkinson's disease. In both Alzheimer's disease and Parkinson's disease ascertainment, the reported AAE was recorded as the age at which study personnel clinically examined a participant. The data description is summarized below in table 2.

TABLE 2

| | VALUE FOR DATA SET | | | | | |
|---|---|---|---|---|---|---|
| CHARACTERISTIC | AD | | PD | | ADPD | |
| No. of families | 449 | | 174 | | 623 | |
| No. of affected individuals | 1,121 | | 378 | | 1,499 | |
| with reported AAO | | | | | | |
| information | | | | | | |
| No. of unaffected individuals | 746 | | 470 | | 1,216 | |
| with reported AAE | | | | | | |
| information | | | | | | |
| Mean ± SD reported AAO, | 72.8 ± 6.8 | | 60.1 ± 12.7 | | 69.6 ± 10.3 | |
| in years (range) | (49 | 97) | (12 | 90) | (12 | 97) |
| Mean ± SD reported AAE, | 70.2 ± 13.0 | | 68.4 ± 12.7 | | 69.6 ± 12.9 | |
| in years, from unaffected | (29 | 105) | (34 | 98) | (29 | 105) |
| individuals (range) | | | | | | |

Overall, for Alzheimer's disease, the study found 1,121 affected individuals with reported AAO and 746 unaffected individuals with reported AAE, and, for Parkinson's disease, 378 affected individuals with reported AAO and 470 unaffected individuals with reported AAE. Average reported AAO±SD was 72.8±6.8 years for Alzheimer's disease and 60.1±12.7 years for Parkinson's disease. Average reported AAE for unaffected individuals was 70.2±13.0 years for Alzheimer's disease and 68.4±12.7 years for Parkinson's disease.

Modeling AAO Data

The AAO data was modeled as suggested by Duggirala et al., (Am J Hum Genet 64:1127-1140 (1999)), where AAO was suggested as a right-truncated quantitative trait in affected individuals (i.e., reported AAO is less than or equal to reported AAE) and a left-truncated quantitative trait in unaffected individuals (i.e., reported AAO is greater than reported AAE). That is, the AAO data was comprised of reported AAO for affected individuals and reported AAE for unaffected individuals. Outliers and normality of the data were examined for agreement with the assumption of QTL analysis. Foul outliers that were 4 SD below the mean AAO were excluded in the Alzheimer's disease data set.

Linkage Analysis

The variance-components procedure in SOLAR was used for linkage analysis. In theory, the quantitative phenotype AAO (y) was defined as a linear function of the n QTLs (rᵢ) that influence the trait:

$$Y = μ + Xβ + {\sum }_{i = 1}^{n} {r}_{i} + ⅇ ,$$

where X is a matrix of covariates and is the regression coefficient matrix associated with the covariates. The phenotype is assumed to follow a normal distribution. The likelihood function of y includes the identical by descent (IBD) probability at a marker that is linked to a QTL, the additive genetic variance attributed by an unobserved QTL (σ_{q}²), and other variance components. SOLAR employs the likelihood-ratio test to test a null hypothesis of σ_{q}²=0 (no linkage) and generates a LOD score that is the equivalent of the classical LOD score of linkage analysis. This technique can be applied to detect the evidence of linkage to an individual marker for two-point analysis or to an imputed chromosomal position in multipoint analysis. Locus-specific IBD information for pairs of relatives was obtained prior to computation of the likelihood function. The multipoint mapping strategy in SOLAR may also be used as an extension of the method of Fulker et al., Am J Hum Genet 56:1224-1233 (1995). It requires the map distance between the markers to create the IBD information of a pair of relatives at a QTL that is linked to a marker. A Kosambi sex-averaged map was used and obtained from Map-O-Mat. The linkage analysis method implemented in SOLAR does not require specification of disease-allele frequency, penetrance, or mode of inheritance, which differs from the classical linkage mapping procedure.

The initial genomic screens were performed on an Alzheimer's disease data set of 449 families with a total of 4,316 relative pairs (sib pairs, cousin pairs, avuncular pairs, etc.) and a Parkinson's disease data set of 174 families with 2,256 relative pairs. A total of 323 (Alzheimer's disease) and 330 (Parkinson's disease) microsatellite markers, with an average spacing of 10 cM, were analyzed. Since AAO was modeled as a truncated quantitative trait, the overall distribution of AAO was thus considered as a mixture of two truncated normal distributions. Both sex and affection status were included as covariates in the polygenic model (model 1), in which affection status was used for adjustment of the contribution of reported AAO and reported AAE to the quantitative trait AAO.

The common regions identified by the initial linkage analyses of Alzheimer's disease and Parkinson's disease data sets were followed up by use of the combined Alzheimer's disease and Parkinson's disease (ADPD) data set for linkage analysis. For the analysis of the ADPD data set, two polygenic models were considered, to incorporate two different scenarios:

Model 1

The assumption of model 1 is that Alzheimer's disease and Parkinson's disease are the same disease, so sex and affection status were included as covariates as that in the initial linkage analysis.

Model 2

In Model 2, the distribution of the ADPD data set was considered to be a mixture of two normal distributions (AAO from Alzheimer's disease and AAO from Parkinson's disease), so that disease status was included as an additional covariate to distinguish the different contribution of AAO between Alzheimer's disease and Parkinson's disease.

Results

Polygenic Models Revealed Strong Heritability of AAO Genies

The analyses of polygenic models showed that sex, affection status, and disease were all significant covariates, for instance, with P values <0.0001 for the ADPD data set. The proportion of variance contributed by all covariates included in the model ranged from 0.9% to 15.3% among different data sets as evidenced in Table 3.

TABLE 3

| | RESIDUAL | | PROPORTION OF | |
|---|---|---|---|---|
| | HERITABILITY^{a} [SE] | | VARIANCE CONTRIBUTED | |
| DATA | (%) | | BY ALL COVARIATES | |
| SET | Model 1 | Model 2 | Model 1 | Model 2 |
| AD | 41.8% [.038] | . . . | 0.9% | . . . |
| PD | 61.3% [.044] | . . . | 15.3% | . . . |
| ADPD | 55.7% [.028] | 49.2% [.029] | 1.8% | 8.3 |

NOTE.
The polygenic models are: model 1, covariate at sex and affection status; and model 2, covariate at sex, affection status, and disease.
^{a}The residual heritability is the ratio of the residual variance (after removing the covariate effects) to the total phenotypic variance.

It was found that the Alzheimer's disease data set demonstrated the smallest proportion of variance from all covariates (0.9%). This may be due to the larger sample size for Alzheimer's disease than for Parkinson's disease or to the marginally significant effect of sex and affection status in Alzheimer's disease (P=0.03 for sex and P=0.05 for affection status). The inheritability of AAO after these covariates were controlled was highly significant in each of the data sets (P<0.0001), with inheritabilities of 42% (Alzheimer's disease), 62% (Parkinson's disease), 56% (ADPD, model 1), and 49% (ADPD, model 2) (table 3). This data strongly indicates that genes are important modulators of AAO.

Disease-Specific Linkage Evidence

The initial linkage analyses were performed on the Alzheimer's disease and Parkinson's disease data sets separately. A threshold of LOD>1.00 was used for declaring a region “interesting” (table 4) and warranting follow-up analyses.

TABLE 4

| CHROMOSOME | MAP | LOD (DISTANCE) IN DATA SET | | | |
|---|---|---|---|---|---|
| AND | POSITION^{a} | | | ADPD | |
| MARKER REGION | (cM) | AD | PD | Model 1 | Model 2 |
| Chromosome 1: | | | | | |
| D1S2134 | 76 | | | | |
| Peak | 78 | | 3.41 | | |
| D1S200 | 82 | | | | |
| Chromosome 4: | | | | | |
| Peak (D4S1652) | 208 | 2.29 | | | |
| Chromosome 5: | | | | | |
| D5S1462 | 105 | | | | |
| Peak | 108 | | 1.65 | | |
| D5S1453 | 115 | | | | |
| Chromosome 6: | | | | | |
| D6S2439 | 43 | | | | |
| Peak | 51 | 1.17 | | | |
| D6S2427 | 54 | | | | |
| Peak (D6S1017) | 63 | | 1.88 | | |
| GATA184A08 | 146 | | | | |
| Peak | 154/156 | | | 1.96 (154 | 1.81 (156 |
| | | | | cM) | cM) |
| D6S1007 | 160 | | | | |
| Chromosome 8: | | | | | |
| D8S1128 | 140 | | | | |
| Peak | 150 | 2.09 | | | |
| D8S373 | 165 | | | | |
| Chromosome 10: | | | | | |
| Peak (D10S1239) | 132 | | 1.55 | | |
| Peak | 133/135 | | | 2.33 (135 | 2.62 (133 |
| | | | | cM) | cM) |
| Peak (D10S1237) | 139 | 2.39 | | | |
| Chromosome 13: | | | | | |
| Peak (D13S800) | 55 | | 1.41 | | |
| Peak (D13S285) | 111 | 1.46 | | | |
| Chromosome 17: | | | | | |
| Peak (D17S1303) | 25 | | 1.93 | | |
| Chromosome 18: | | | | | |
| Peak (D18S877) | 54 | 1.33 | | | |
| Chromosome 20: | | | | | |
| D20S851 | 25 | | | | |
| Peak | 27 | | 1.47 | | |
| D20S604 | 33 | | | | |
| Chromosome 22: | | | | | |
| Peak (D22S683) | 37 | | 1.32 | | |

Of greatest interest for Parkinson's disease is the result on chromosome 1, near D1S2134 (78 cM; LOD=3.41). For Alzheimer's disease, examination of APOE, a known modulator of AAO in Alzheimer's disease, generated a LOD score of 3.28 in the Alzheimer's disease data set, confirming Its role as a modulator of AAO. In addition to APOE, strong AD-specific suggestive linkage regions on chromosome 4q at D4S 1652 (208 cM; LOD=2.29) and chromosome 8q (150 cM, LOD=2.09) were also found. Interestingly, neither chromosome 4q nor chromosome 8q have previously been reported as linkage regions with Alzheimer's disease risk genes. It is possible that chromosomes 4q and 8q harbor genes that exclusively modulate onset of Alzheimer's disease.

Linkage Evidence for a Common AAO Gene on Chromosome 10q

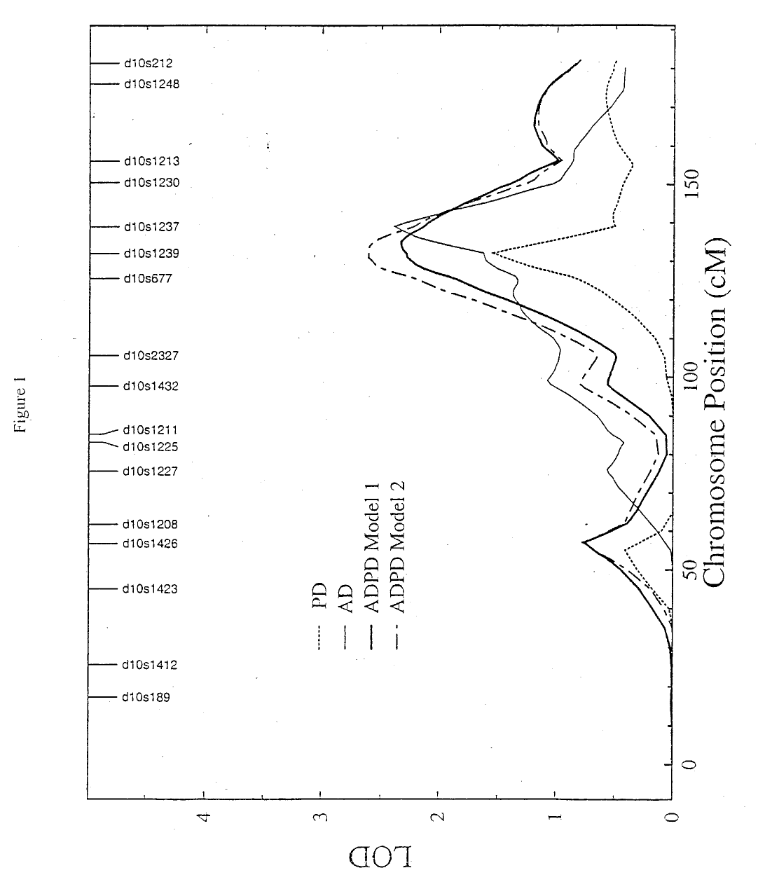

In the initial genomic screens, chromosomes 6 and 10 gave evidence for linkage to AAO in both the Alzheimer's disease and Parkinson's disease data sets (see table 4). The peaks were 12 cM apart on chromosome 6 (51 cM in Alzheimer's disease and 63 cM in Parkinson's disease) and were 7 cM apart on chromosome 10 (132 cM in Parkinson's disease and 139 cM in Alzheimer's disease). To decipher the role of these common interesting linkage regions, linkage analyses were performed on the combined ADPD data set for chromosomes 6 and 10. Analysis of the ADPD data set by use of models 1 and 2 did not result in a consistent area of interest for chromosome 6, as the combined data set gave peak LOD scores of 1.96 at 154 cM for model 1 and 1.81 at 156 cM for model 2. This peak from the combined ADPD data set is unlinked to both the independent Alzheimer's disease (51 cM) and PD (63 cM) regions. However, analysis of the ADPD data set on chromosome 10 confirmed the findings from the analyses of the independent Alzheimer's disease and Parkinson's disease data sets, resulting in a single peak region on chromosome 10q between D10S1239 and D10S1237, with LOD scores of 2.33 for model 1 at 133 cM and 2.62 for model 2 at 135 cM (table 4). FIG. 1 summarizes the multipoint results of the four analyses on chromosome 10. As can be seen, four analyses revealed a similar pattern of LOD scores across chromosome 10, with a single peak region. Notably, inclusion of disease status as a covariate in model 2 slightly increases the LOD score in the ADPD data set. The present results suggest that a common modulator of AAO for Alzheimer's disease and Parkinson's disease may be located on chromosome 10q.

Glutathione S-Transferases, Omega-1 (GSTO1)

Glutathione S-Transferases, Omega-1 (GSTO1), deposited as accession number AF212303 (from OMIM at http://www.ncbi.nlm.nih.gov/) is a member of a family of enzymes that use glutathione in reactions that contribute to the transformation of many compounds, including therapeutic drugs, carcinogens, and the products of oxidative stress. GSTO1 (Board et al. 2000) is located on chromosome 10q24.3)2, which is very close to the peak linkage marker (3 cM apart from D10S1237) for common age at onset (AAO) genes for Alzheimer (AD) and Parkinson (PD) diseases. The deduced 241-amino acid protein of GSTO1 has over 70% identity with the rodent omega GST sequence. GSTO1 is expressed in a wide range of tissues including liver, skeletal muscle, and heart, brain, placenta, and lung. GSTO1 lacks activity with most GST substrates but has high thiol transferase activity. The wide spread expression and highly conserved sequence of GSTO1 indicates that it has significant several functions, including protection from oxidative stress.

Expression Studies

Expression analysis was performed microarray gene on two controls and 6 AD patients. The cRNA was insolated and hybridized to microarray chip (Affymetrix). The intensity of all features of microarrays were recorded and examined for artifacts using Affymetrix GeneChip® Software v 4.0, according to standard Affymetrix procedures (O'Dell et al. 1999). The raw expression levels are then multiplied by a scaling factor to make the mean expression level on the microarray equal to a “target intensity” of 100. The Affymetrix software to normalize the gene expression levels to allow comparison between any two groups of samples automatically performs this scaling. GSTO1 was down regulated in RNA transcript of AD patients and up regulated in controls through hierarchical cluster analysis. Similar changes have been seen in GSTO1 in the SAGE analysis of PD patients and controls.

Association Analysis

The IMS-JST091371 SNP (GSTO1EX4CA1, JSNP database) was genotyped in 713 AD and 284 PD multiplex families. It corresponds to a C to A base pair change (GCT>GAT) at position 53 in the exon 4 of GSTO1, which causes a non-conservative residue change from an alanine (hydrophobic amino acid) to an aspartic acid (hydrophilic and acidic amino acid). The SNP was genotyped using a TaqMan assay with the following primers and probes:

5′-TGTCTAGGTGCCATCCTTGGT-3′,

5′-TCCTCTAGCTTGGTAAATTCTTTACGA-3′,

6FAM-AAGACTATGCTGGCCTA-TAMGBNFQ,

VIC-AAGACTATGCTGGCCTAA-TAMGBNFQ.

The variance component model was used as described in Abecasis et al. (2000) to test the association between the SNP and AAO of AD and PD, respectively. The variance component method allows simultaneously modeling the mean and variance, so that all information in a set of related individuals can be used to construct a test of association. Therefore, this method is more appropriate for multiplex families used in the present study. This method has been implemented in QTDT program (http://www.sph.umich.edu/csg/abecasis/QTDT/index.html). GSTO1EX4CA1 was found to be significantly associated with AAO in AD in the overall data set with p-value=0.009 and in PD with p-value=0.05 overall and p-value=0.03 in the family history+PD (Table 5). Joint analysis of the AD/PD dataset gave evidence of significant association with a p-value=0.001 in the late-onset PD and late-onset PD with a positive family history (Table 5). Thus, both statistical and molecular data strongly support that GSTO1 plays a significant biological role in the control of age at onset of AD and PD.

TABLE 5
GSTO1 Results

| | | Nominal p-value (Empirical p-value) | | |
|---|---|---|---|---|
| Disease | Data set | # family | Orthogonal Model | Fulker Model |
| AD | All data | 713 | 0.009 (0.006) | 0.01 (0.006) |
| | All ind with AAO | 691 | 0.014 | 0.014 |
| | >65* | | | |
| | Late Onset Families | 516 | 0.014 (0.008) | 0.014 (0.008) |
| AD | selected family** | 611 | 0.01 | 0.006 |
| PD | Multiplex Families | 284 | 0.053 | 0.054 |
| | Multiplex & Singleton | 661 | 0.068 (0.052) | 0.127 (0.038) |
| | Late Onset | 581 | 0.026 | 0.084 |
| | Fam History + | 303 | 0.028 (0.023) | 0.023 (0.019) |
| ADPD | Late Onset Fam | 269 | 0.012 (0.005) | 0.018 (0.012) |
| | History + | | | |
| | AD + | 997 | 0.007 | 0.007 |
| | multiplex PD | | | |
| | AD + all PD | 1374 | 0.016 (0.005) | 0.044 (0.013) |
| | AD and PD+ | 1016 | 0.004 (0.004) | 0.004 (0.004) |
| | Late Onset ADPD | 1097 | 0.004 (0.002) | 0.017 (0.009) |
| | Late Onset AD + | 785 | 0.001 (0.001) | 0.002 (0.001) |
| | Late Onset PDfamP | | | |

NGSTO

Additionally, a similar gene, NGSTO, was examined in the data. NGSTO maps adjacent to GSTO and may represent an ancestral duplication of the GSTO gene. NGSTO also showed evidence of association (Table 6). Table 7 gives the haplotype analysis of the GSTO1 and NGSTO genes. In conclusion, these linkage, association and expression data support the involvement of the GSTO1 gene and related genes in AAO in AD and PD.

TABLE 6
NGSTO Results

| | | | | | p-value (emperical p) | |
|---|---|---|---|---|---|---|
| Disease | DATA | # of fam | Marker | Orthogonal | Fulker | Monks |
| AD | Overall AD | 713 | ngsto683 | 0.024 | 0.014 | |
| | LOAD | 516 | ngsto683 | 0.02 | 0.02 | 0.059 |
| AD | selected fam | 611 | ngsto683 | 0.014 | 0.008 | |
| PD | Overall PD | 611 | ngsto683 | 0.076 | | |
| | FamHP+ | 303 | ngsto683 | 0.076 (0.063) | | |

TABLE 7
Haplotype Results NGSTO and GSTO

| AD | |
|---|---|
| Total ind | 972 |

| | | | |
|---|---|---|---|
| Two Loci | | | |
| global-stat = 6.92143 | df = 3 | p-val = | 0.07444 |

| | gsto | ngsto683 | Hap-Freq | Hap-Score | p-val |
|---|---|---|---|---|---|
| [1] | 1 | 2 | 0.22948 | −2.61813 | 0.00884 |
| [2] | 1 | 1 | 0.01714 | 0.41546 | 0.67781 |
| [3] | 2 | 1 | 0.75285 | 2.41061 | 0.01593 |

| | | | |
|---|---|---|---|
| Three Loci | | | |
| global-stat = 7.6472 | df = 4 | p-val = | 0.10539 |

| | gsto | ngsto683 | ngsto684 | Hap-Freq | Hap-Score | p-val |
|---|---|---|---|---|---|---|
| [1] | 1 | 2 | 1 | 0.23044 | −2.74527 | 0.00605 |
| [2] | 1 | 1 | 2 | 0.01691 | 0.39806 | 0.69058 |
| [3] | 2 | 1 | 1 | 0.18922 | 0.90755 | 0.36412 |
| [4] | 2 | 1 | 2 | 0.5629 | 1.40248 | 0.16077 |

| | | | |
|---|---|---|---|
| Four Loci | | | |
| global-stat = 12.22057 | df = 5 | p-val = | 0.03189 |

| | gsto | ngsto683 | ngsto684 | ngstoex3 | Hap-Freq | Hap-Score | p-val |
|---|---|---|---|---|---|---|---|
| [1] | 1 | 2 | 1 | 2 | 0.23201 | −2.85239 | 0.00434 |
| [2] | 2 | 1 | 1 | 1 | 0.09667 | −1.01954 | 0.30795 |
| [3] | 1 | 1 | 2 | 1 | 0.01719 | 0.33883 | 0.73474 |
| [4] | 2 | 1 | 2 | 1 | 0.56069 | 1.51507 | 0.12976 |
| [5] | 2 | 1 | 1 | 2 | 0.09291 | 2.12151 | 0.03388 |

Materials and Methods

Microarray Gene Expression Study:

Total RNA was extracted using TRIzol reagent (Invitrogen, Carlsbad, Calif.) according to the manufacturer's instructions. To label the RNA for hybridization to the microarray chip, 7 μg of total RNA were used for double-stranded cDNA synthesis using the SuperScript Choice System (Gibco BRL Life Technologies, Rockville, Md.) in conjunction with a T7-(dT)-24 primer (Geneset Oligos, La Jolla, Calif.). The cDNA was purified using Phase Lock Gel (3 Prime, Inc., Boulder, Colo.). In vitro transcription was performed to produce biotin-labeled cRNA using a BioArray HighYield RNA Transcript Labeling Kit (Affymetrix, Santa Clara, Calif.) according to the manufacture's instructions. The biotinylated RNA was cleaned using the RNeasy Mini kit (Qiagen, Valencia, Calif.). See, Lockhart et al., Nat. Biotechnol. 14, 1675 (1996); and Warrington et al., Physiol Genomics 2, 143 (2000).

To probe the microarray, 20 μg of biotinylated cRNA was fragmented and hybridized to microarrays (GeneChip Human Genome U133A array, Affymetrix) using previously described protocols. See, Lockhart et al. The intensity of all features of microarrays was recorded and examined for artifacts (Affymetrix GeneChip® Software v 4.0). O'Dell et al., Eur. J. Hum. Genet 7, 821 (1999). Quantitative gene expression values measured by the average difference between the hybridization intensity with the perfect match probe sets and the mismatch probe sets were then multiplied by a scaling factor to make the mean expression level on the microarray equal to a target intensity of 100. The Affymetrix software to normalize the gene expression levels automatically performs this scaling.

For quality control, all arrays were visually inspected to exclude hybridization artifacts. To control for partial RNA degradation, 3′/5′ end ratios for the housekeeping genes actin and GAPDH were examined. Arrays with high 3′/5′ end ratios suggestive of partial RNA degradation were excluded from further analysis.

Microarray Data Analysis:

Since genes with low signal intensity often cause high variability between arrays and Northern blots usually do not confirm positive results for genes with signal intensity less than 500, only genes with average expression intensities of =500 were considered for further analysis. A log₂ (logarithm base 2) was used for data normalization, so data within each chip are in agreement with normal distribution. A two-sample t-test was used to examine whether the gene expression between case and control groups is significantly different Disease status was randomly assigned to each sample for 1000 times to estimate an empirical p-value for each gene. A nominal significance level of 0.05 was compared with the empirical p-values to declare a result significant.

SNP Detection and Genotyping:

Public domain databases (Japanese JSNP, http://snp.ims.u-tokyo.ac.ip, NCBI dbSNP, http://www.ncbi.nlm.nih.gov/SNP/, and Applied Biosystems http://www.appliedbiosystems.com) were utilized to identify SNPs located in or near the five candidate genes. Two SNPs in GSTO1 (SNP6 and SNP7), three SNPs in GSTO2 (SNP8-10), and SNP14 in PRSS11 were genotyped using the primers and probes listed in Table S2.

TABLE S2

Primers and probes used for SNP genotyping. The position and the
base pair/amino acid change of each SNP are indicated.

| | | Position Chr. 10 | |
|---|---|---|---|
| | SNP | [NCBI Build 31] | |
| Gene | dbSNP# | (Change) | Primers and probes sequence (5′-3′) |
| SCD | SNP1 | 101 347 325 | |
| | rs3870747 | (1VS3 − 505 C > T) | C 1345738_10 ABI Assay-on-demand |
| | | 101 355 833 | C_11743455_10 ABI Assay-on-demand |
| | SNP2 | (3′UTR + 1497 C > G) | |
| NDUFB8 | SNP3 | 101 522 724 | |
| | rs1800662 | (1VS3 + 109 A > C) | C_8866465_1 ABI Assay-on-demand |
| | | 105 241 160 | |
| GSTO2 | SNP4 | (−7155 C > T) | C_2952474_10 ABI Assay-on-demand |
| | SNP5 | 105 24 7082 | C 2086928_10 ABI Assay-on-demand |
| | rs2164624 | (−1233 A > G) | |
| | | | TGTCTAGGTGCCATCCTTGGT |
| | SNP6* | 105 256 426 | TCCTCTAGGTTGGTAAATTCTTTACGA |
| | rs4925 | (Ala140Asp) | FAM-AAGACTATGCTGGCCTA-MGBFNQ |
| | | | VIC-AAGACTATGATGGCCTAA-MGBFNQ |
| | | | GCTGCAGTGAACATTCACATAACAT |
| | SNP7 | 105 258 895 | TGGATACTCATCACCCAGCAAT |
| | rs1147611 | (1VS4 − 584 G > T) | VIC-ACTTGGCAATGTAAC-MGBNFQ |
| | | | FAM-TGTACTTGGAAATGTAAC-MGBNFQ |
| | | | ACTCTCGGGCTTCCAAATCTG |
| GSTO2 | SNP8 | 105 268 128 | GCGATCTGGAGCAGGAGCTA |
| | rs2297235 | (−183 C > T) | FAM-CCCAGGTTAAGTTAC-MGBNFQ |
| | | | VIC-CCAGGTTAAATTAC-MGBNFQ |
| | | | GCCAAAAGATGTTATTGGAGCTATTT |
| | SNP9 | 105 271 531 | TTGGGAAAGACATGCAAAGTAAAAT |
| | rs157077 | (1VS3 + 20 G > A) | FAM-TGTGAGTGGCTTTT-MGBFNQ |
| | | | VIC-CAGTGTGAGTGAGTTT-MGBFNQ |
| | | | GCCTGGTAGCGTTGAGATGTG |
| | SNP10 | 105 272 822 | TTTTGTACCTCTTCCAGGTTGCT |
| | rs156697 | (Asn142Asp) | FAM-AGAATGCACTAATCTGAAGGCAGCCC-BHQ1 |
| | | | TET-AGAATGCACTGATCTGAAGGCAGCC-BHQ1 |
| PRSS11 | SNP11 | 123 480 796 | |
| | | (1VS1 − 10806 A > G) | C_2761707_10 ABI Assay-on-demand |
| | SNP12 | 123 499 529 | |
| | rs714816 | (1VS3 + 7203 G > A) | C_2347168_1 ABI Assay-on-demand |
| | SNP13 | 123 505 603 | |
| | rs2250511 | (1VS3 − 3788 A > G) | C 2761733_1 ABI Assay-on-demand |
| | | | CATGTAAAGTCAGACCAGGAGGAA |
| | SNP14 | 123 516 855 | TGCAACACAAAGGGAAACACA |
| | rs2293871 | (1VS8 − 36 T > C) | VIC-TGGAAACATGAAACAT-MGBFNQ |
| | | | FAM-AAACACGAAACATTG-MGBFNQ |

*SNPs genotyped in both Alzheimer's disease and Parkinson's disease datasets.

All other SNPs were genotyped using the assays-on-demand from Applied Biosystems (ABI, Foster City, Calif.). All 14 SNPs were genotyped in Alzheimer's disease datasets. As a result of the association analysis, SNP6 and SNP8 were also examined in the Parkinson's disease dataset. Genomic DNA was extracted form whole blood using the PureGene system (Gentra Systems, Minneapolis, Minn.) and genotyped using the TaqMan allelic discrimination assay. Apolipoprotein E (APOE) genotyping was performed as previously described. See, Saunders et al., Neurol. 43, 1467 (1993); and Vance et al., Approaches to Gene Mapping in Complex Human Diseases, (Wiley-Liss, New York, 1998), chap. 9.

Association Analysis:

All SNPs were tested for Hardy-Weinberg equilibrium (HWE) and linkage disequilibrium (LD) in the affected group (one affected from each family) and the unaffected group (one unaffected from each family). An exact test implemented in Genetic Data Analysis (GDA) program was used to test HWE, in which 3,200 replicate samples were simulated for estimating the empirical P value. See, Zaykin et al., Genetica, 96, 169 (1995). The GOLD (Graphical Overview of Linkage Disequilibrium) program was used to estimate the Pearson correlation (r²) of alleles for each pair of SNPs as the measurement of LD. See, Abecasis et al. The higher the r² (0<r²<1), the stronger the LD. In general, r²>0.3 is considered to be a minimum useful value for detecting association with an unmeasured variant related to disease risk by genotyping a nearby marker in LD with that variant. See, Ardlie et al., Nat. Rev. Genet. 3, 299 (2002).

The orthogonal model takes information from a general pedigree. It can incorporate covariate effects when necessary. The association between the marker and age-at-onset was identified by testing within family effect, which is equivalent to the additive effect of the marker locus. The empirical p-values were computed through 1000 permutations to avoid false-positive results.

In the specification, there has been disclosed typical preferred embodiments of the invention and, although specific terms are employed, they are used in a generic and descriptive sense only and not for purposes of limitation of the scope of the invention being set forth in the following claims.

What is claimed is:

1. A method of screening a subject for Alzheimer's disease and/or Parkinson's disease comprising:
detecting the presence or absence of at least one or more markers linked to Alzheimer's disease and or Parkinson's disease, wherein the presence of said marker indicates that the subject is afflicted with or at risk of developing Alzheimer's disease and or Parkinson's disease, and wherein said marker is selected from the group consisting of D4S1652, D10S1239, D10S1237, D1S2134, D8S1128, D8S373, D1S200, D5S1462, D5S1453, D6S2439, D6S2427, D6S1017, D6S1007, D13S800, D13S285, D17S1303, D18S877, D20S851, D20S604, D22S683 and markers within fifteen centimorgans thereof.

2. The method according to claim 1, wherein said marker is linked to age of onset of Alzheimer's disease and/or Parkinson's disease.

3. The method according to claim 1, wherein said Alzheimer's disease is late-onset Alzheimer's disease.

4. The method according to claim 1, wherein said method is a diagnostic method.

5. The method according to claim 1, wherein said method is a prognostic method.

6. The method according to claim 1, wherein said Parkinson's disease is early-onset Parkinson's disease.

7. The method according to claim 1, wherein said subject is human.

8. A method for diagnosing a subject as having Alzheimer's disease and/or Parkinson's disease, or as having a predisposition to Alzheimer's disease and/or Parkinson's disease comprising:
determining the presence or absence of an allele of a polymorphic marker in the subject, wherein (i) the allele is associated with a phenotypic marker of Alzheimer's disease and/or Parkinson's disease, and wherein (ii) the polymorphic marker is within a segment selected from the group consisting of:
a segment of chromosome 1 bordered by D1S2134 and D1S200;
a segment of chromosome 4 within 2 centimorgans of D4S1652;
a segment of chromosome 5 bordered by D5S1462 and D5S1453;
a segment of chromosome 6 bordered by D6S2439 and D6S2427;
a segment of chromosome 6 bordered by D6S 1017 and D6S 1007;
a segment of chromosome 8 bordered by D8S1128 and D8S373;
a fifteen centimorgan linkage region surrounding D10S1239 and D10S1237;
a segment of chromosome 13 bordered by D13S800 and D13S285;
a segment of chromosome 17 bordered by D17S1303 and D18S877;
a segment of chromosome 20 bordered by D20S851 and D20S604; and
a segment of chromosome 22 within 2 centimorgans of D22S683.

9. The method according to claim 8, wherein said determining the presence or absence of an allele of a polymorphic marker in the subject is performed utilizing DNA or RNA.

10. The method according to claim 8, wherein said marker is linked to age of onset of Alzheimer's disease and/or Parkinson's disease.

11. The method according to claim 8, wherein said Alzheimer's disease is late-onset Alzheimer's disease.

12. The method according to claim 8, wherein said method is a diagnostic method.

13. The method according to claim 8, wherein said method is a prognostic method.

14. The method according to claim 8, wherein said Parkinson's disease is early-onset Parkinson's disease.

15. The method according to claim 8, wherein said subject is human.

16. An oligonucleotide primer for amplification of an allele which is associated with Alzheimer's disease and/or Parkinson's disease, wherein said allele is located at a locus in a region selected from the group consisting of:
a segment of chromosome 1 bordered by D1S2134 and D1S200;
a segment of chromosome 4 within 2 centimorgans of D4S1652;
a segment of chromosome 5 bordered by D5S1462 and D5S1453;
a segment of chromosome 6 bordered by D6S2439 and D6S2427;
a segment of chromosome 6 bordered by D6S1017 and D6S1007;
a segment of chromosome 8 bordered by D8S1128 and D8S373;
a fifteen centimorgan linkage region surrounding D10S1239 and D10S1237;
a segment of chromosome 13 bordered by D13S800 and D13S285;
a segment of chromosome 17 bordered by D17S1303 and D8S877;
a segment of chromosome 20 bordered by D20S851 and D20S604; and
a segment of chromosome 22 within 2 centimorgans of D22S683.

17. The oligonucleotide primer of claim 16, wherein said primer is from 5 to 50 nucleotides in length.

18. The oligonucleotide primer of claim 16, wherein said allele is linked to age of onset of Alzheimer's disease and/or Parkinson's disease.

19. The oligonucleotide primer of claim 16, wherein said Alzheimer's disease is late-onset Alzheimer's disease.

20. The oligonucleotide primer of claim 16, wherein said Parkinson's disease is early-onset Parkinson's disease.

21. An assay for detecting a gene related to an age of onset disorder comprising:
providing a biological sample comprising genomic DNA from a patient suspected of having or at risk for developing said age of onset disorder;
using a probe directed toward to a region of a polymorphic marker in the subject, wherein (i) the marker is associated with a phenotypic marker of Alzheimer's disease and/or Parkinson's disease, and wherein (ii) the polymorphic marker is within a segment selected from the group consisting of:
a segment of chromosome 1 bordered by D1S2134 and D1S200;
a segment of chromosome 4 within 2 centimorgans of D4S1652;
a segment of chromosome 5 bordered by D5S1462 and D5S1453;
a segment of chromosome 6 bordered by D6S2439 and D6S2427;
a segment of chromosome 6 bordered by D6S 1017 and D6S1007;
a segment of chromosome 8 bordered by D8S1128 and D8S373;
a fifteen centimorgan linkage region surrounding D10S1239 and D10S1237;
a segment of chromosome 13 bordered by D13S800 and D13S285;
a segment of chromosome 17 bordered by D17S1303 and D18S877;
a segment of chromosome 20 bordered by D20S851 and D20S604; and
a segment of chromosome 22 within 2 centimorgans of D22S683; and
detecting duplications in the region of the genomic sequence of the group of chromosomes listed above.

22. The assay of claim 21, where said age of onset disease is Alzheimer's disease and/or Parkinson's disease.

23. The assay of claim 21, wherein said Alzheimer's disease is late-onset Alzheimer's disease

24. The assay of claim 21, wherein said Parkinson's disease is early-onset Parkinson's disease.

25. A method for diagnosing a subject as having Parkinson's disease, or as having a predisposition to Parkinson's disease comprising:
determining the presence or absence of an allele of a polymorphic marker in the subject, wherein (i) the allele is associated with a phenotypic marker of Parkinson's disease, and wherein (ii) the polymorphic marker is within a segment of chromosome 1 within 2 centimorgans of D1S2134.

26. The method according to claim 25, wherein said determining the presence or absence of an allele of a polymorphic marker in the subject is performed utilizing DNA or RNA.

27. The method according to claim 25, wherein said allele is linked to age of onset of Parkinson's disease.

28. The method according to claim 25, wherein said method is a diagnostic method.

29. The method according to claim 25, wherein said method is a prognostic method.

30. The method according to claim 25, wherein said Parkinson's disease is early-onset Parkinson's disease.

31. The method according to claim 25, wherein said subject is human.

32. A method for diagnosing a subject as having Alzheimer's disease or Parkinson's disease, or as having a predisposition to Alzheimer's disease or Parkinson's disease comprising:
determining the presence or absence of an allele of a polymorphic marker in the subject, wherein (i) the allele is associated with a phenotypic marker of Alzheimer's disease or Parkinson's disease, and wherein (ii) the polymorphic marker is within a fifteen centimorgan linkage region surrounding D10S1239 and D10S1237.

33. The method according to claim 32, wherein the determining the presence or absence of an allele of a polymorphic marker in the subject comprises detecting a marker in GSTO1.

34. The method according to claim 32, wherein the determining the presence or absence of an allele of a polymorphic marker in the subject comprises detecting a marker in GSTO2.

35. A method for diagnosing a subject as having Alzheimer's disease, or as having a predisposition to Alzheimer's disease comprising:
determining the presence or absence of an allele of a polymorphic marker in the subject, wherein (i) the allele is associated with a phenotypic marker of Alzheimer's disease, and wherein (ii) the polymorphic marker is within a segment of chromosome 4 within 2 centimorgans of D4S1652.

36. The method according to claim 33, wherein said determining the presence or absence of an allele of a polymorphic marker in the subject is performed utilizing DNA or RNA.

37. The method according to claim 33, wherein said allele is linked to age of onset of Alzheimer's disease.

38. The method according to claim 33, wherein said Alzheimer's disease is late-onset Alzheimer's disease

39. The method according to claim 33, wherein said method is a diagnostic method.

40. The method according to claim 33, wherein said method is a prognostic method.

41. The method according to claim 33, wherein said subject is human.

42. A method of determining quantitative trait age of onset for Alzheimer's disease and/or Parkinson's disease comprising:
utilizing the variance component procedure in SOLAR to perform a genomewide scan on the quantitative trait age of onset for Alzheimer's disease and/or Parkinson's disease;
accounting for covariate and/or random effects; and
mapping quantitative trait loci influencing age of onset.

43. The method according to claim 42, wherein said allele is linked to age of onset of Alzheimer's disease and/or Parkinson's disease.

44. A computer assisted method of identifying a proposed treatment for Alzheimer's disease and/or Parkinson's disease comprising:
storing a database of biological data for a plurality of patients, the biological data including for each of said plurality of patients (i) a treatment type, (ii) at least one genetic marker associated with Alzheimer's Disease and/or Parkinson's disease, and (iii) at least one disease progression measure for Alzheimer's Disease and/or Parkinson's disease from which treatment efficacy may be determined; and
querying said database to determine the dependence on said genetic marker of the effectiveness of a treatment type in treating Alzheimer's disease and/or Parkinson's disease, to thereby identify a proposed treatment as an effective treatment for a patient carrying a particular marker for Alzheimer's disease and/or Parkinson's disease.

45. The method according to claim 44, wherein said marker is linked to age of onset of Alzheimer's disease and/or Parkinson's disease.

46. The method according to claim 44, wherein said Alzheimer's disease is late-onset Alzheimer's disease.

47. The method according to claim 44, wherein said Parkinson's disease is early-onset Parkinson's disease.

48. A method of screening a subject for Alzheimer's disease and/or Parkinson's disease comprising detecting the presence or absence of at least one or more genes linked to Alzheimer's disease and or Parkinson's disease, wherein the presence of said gene indicates that the subject is afflicted with or at risk of developing Alzheimer's disease and or Parkinson's disease, and wherein said gene is GSTO1 or GSTO2.

49. The method according to claim 48, wherein said gene is linked to age of onset of Alzheimer's disease and/or Parkinson's disease.

50. A method of screening a subject for Alzheimer's disease and/or Parkinson's disease comprising detecting the presence or absence of at least one or more enzymes linked to Alzheimer's disease and or Parkinson's disease, wherein the presence of said enzyme indicates that the subject is afflicted with or at risk of developing Alzheimer's disease and or Parkinson's disease, and wherein said enzyme is GSTO1 or GSTO2.

51. The method according to claim 50, wherein said enzyme is linked to age of onset of Alzheimer's disease and/or Parkinson's disease.

52. A method of screening a subject for Alzheimer's disease and/or Parkinson's disease comprising detecting the presence or absence of at least one or more markers linked to Alzheimer's disease and or Parkinson's disease, wherein the presence of said marker indicates that the subject is afflicted with or at risk of developing Alzheimer's disease and or Parkinson's disease, and wherein said marker is located on chromosome 10q24.32.

53. A method of screening a subject for Alzheimer's disease and/or Parkinson's disease comprising detecting the presence or absence of mutations within the chromosome 10q linkage region selected from the group of genes consisting of: Stearoyl-CoA desaturase (SCD), NADH-ubiquinone oxidoreductase 1 beta complex, 8 (NDUFB8), glutathione S-transferase, omega-1 (GSTO1), and protease, serine 11 (PRSS11).

54. The method according to claim 53, wherein said detecting the presence or absence of genes comprises a single nucleotide polymorphism wherein a non-conservative amino acid is changed from a hydrophobic residue to a hydrophilic residue.

55. The method according to claim 53, wherein said detecting the presence or absence of genes comprises detecting the presence or absence of a mutation at position 140 in the GSTO1 gene.

56. The method according to claim 55, wherein said mutation is Ala140Asp.

57. The method according to claim 53, wherein said detecting the presence or absence of genes comprises detecting the presence or absence of a mutation at position 142 in the GSTO2 gene.

58. The method according to claim 57, wherein said mutation is Asn142Asp.
